(19) United States
(12) Patent Application Publication
(10) Pub. No.: US 2008/0182244 A1
(43) Pub. Date: Jul. 31, 2008

(54) Pre-Implantation Genetic Diagnosis Test

(75) Inventors: Triantafyllos Tafas, Rocky Hill, CT (US); Ilia Ichetovkin, Hamden, CT (US); Michael Kilpatrick, West Hartford, CT (US); Petros Tsipouras, Madison, CT (US)

Correspondence Address: KELLEY DRYE & WARREN LLP, 400 ALTLANTIC STREET , 13TH FLOOR, STAMFORD, CT, 06901, US

(73) Assignee: Ikonisys, Inc., New Haven, CT (US)

(21) Appl. No.: 11/833,678

(22) Filed: Aug. 3, 2007

Related U.S. Application Data
(60) Provisional application No. 60/821,554, filed on Aug. 4, 2006.

(51) Int. Cl. C12Q 1/68

(52) U.S. Cl. 435/6

(57) ABSTRACT

A method for determining viable normal blastomeres for implantation entailing labeling the blastomere with an antibody to hyperglycoslyated hCG and determining the binding of chromosomal probes directed to chromosomal regions of the chromosome.

### CROSS-REFERENCE TO RELATED APPLICATIONS

This application claims the benefit of priority of U.S. Provisional Application No. 60/821,554 filed Aug. 4, 2006, which is incorporated by reference herein in its entirety.

### BACKGROUND OF THE INVENTION

All references cited in this specification, and their references, are incorporated by reference herein where appropriate for teachings of additional or alternative details, features, and/or technical background.

1. Field of the Invention

The present invention generally relates to method for improving pre-implantation genetic diagnosis by using an anti-hyperglycosylated hCG antibody in conjunction with chromosome probes.

2. Description of the Related Art

Pre-implantation genetic diagnosis (PGD) is an early form of prenatal diagnosis that consists of the performance of a genetic test on oocytes prior to fertilization or embryos before they are implanted in the uterus. PGD involves examination of the chromosomes contained in the polar body, taken from an egg, or a blastomere from a developing embryo. Embryonic PGD involves screening embryos created during an in vitro fertilization (IVF) cycle before they are returned to the uterus. A single cell is removed from the embryo and the genetic material is examined to screen for abnormalities. See “Pre-implantation Genetic Diagnosis”, Harper J C, Delhanty J D A and Handyside A H (eds.), Wiley Interscience, (2002) for a discussion of PGD.

Human chorionic gonadotropin (hCG) is a hormonal glycoprotein elaborated by an embryo and subsequently by the placenta at early stages of pregnancy. The protein is a heterodimer, composed of an alpha subunit which is common to several other protein hormones, and a unique hCG beta subunit. The hormone stimulates the corpus luteum to secrete progesterone, which then acts on the uterus to sustain the growth of the embryo. The glycosylation pattern on hCG varies over time during pregnancy (Kovalevskaya et al., J Endocrin 172, 497-506 (2002)). Hyperglycosylated hCG is the principal hCG-related molecule synthesized by choriocarcinoma cytotrophoblast cells and placental cytotrophoblast cells at the time of implantation. It has been indicated to be a promoter of invasive activities in choriocarcinoma cells, and in cytotrophoblast cells isolated from early pregnancy placenta.

PGD is particularly useful for couples with a known history of genetic disease, this early screening of embryos allows only embryos without known genetic defects to be returned to the uterus. This can be a monogenic disorder (autosomal recessive, autosomal dominant or X-linked disorders) or a chromosomal structural aberration (such as a balanced translocation). Nonlimiting examples of monogenic disorders include cystic fibrosis, Beta-thalassemia, sickle cell disease and spinal muscular atrophy type 1 mmyotonic dystrophy, Huntington's disease and Charcot-Marie-Tooth disease; and in the case of the X-linked diseases fragile X syndrome, haemophilia A and Duchenne muscular dystrophy. In the case of chromosomal abnormalities, PGD is mainly carried out for reciprocal and Robertsonian translocations, and in a few cases for other abnormalities such as chromosomal inversions or deletions. Other diseases which may be screened out with this procedure include Down Syndrome (Trisomy 21), Cri du Chat (Trisomy 18), and Tay Sachs disease. It has also been proposed for patients with obstructive and non-obstructive azoospermia.

PGD can be performed on cells from different developmental stages; the biopsy procedures vary accordingly. In general, a biopsy can be performed at all pre-implantation stages. Conventionally the biopsy is performed on unfertilised and fertilised oocytes (for polar bodies, PBs), on day three cleavage-stage embryos (for blastomeres) and on blastocysts (for trophectoderm (TE) cells). Biopsy conventionally entails two steps: first, the opening of the zona pellucida, and second, the removal of the cell(s). There are different approaches to both steps, including mechanical, chemical (Tyrode's acidic solution) and laser technology for the breaching of the zona pellucida, extrusion or aspiration for the removal of PBs and blastomeres, and herniation of the trophectoderm cells.

Fluorescent in situ hybridization (FISH) and polymerase chain reaction (PCR) are the two most commonly used technologies in PGD, although other approaches have been proposed or are currently in development (such as whole genome amplification and comparative genomic hybridization). PCR is generally used to diagnose monogenic disorders and FISH is used for the detection of chromosomal abnormalities (for instance, aneuploidy screening or chromosomal translocations).

FISH, in contrast to karyotyping, can be used on interphase chromosomes, so that it can be used on PBs, blastomeres and TE samples. The cells are fixed on glass microscope slides and hybridised with DNA probes. Each of these probes is specific for part of a chromosome, and each is labelled with a fluorochrome. Currently, a large panel of probes are available for different segments of all chromosomes, but the limited number of different fluorochromes confines the number of signals that can be analysed simultaneously. The type and number of probes that are used on a sample depends on the indication. The use of probes for chromosomes X, Y, 13, 14, 15, 16, 18, 21 and 22 has the potential of detecting 70% of the aneuploidies found in spontaneous abortions. In order to be able to analyse more chromosomes on the same sample, up to three consecutive rounds of FISH can be carried out. In the case of chromosome rearrangements, specific combinations of probes have to be chosen that flank the region of interest. The FISH technique is considered to have an error rate between 5 and 10%.

The main problem of the use of FISH to study the chromosomal constitution of embryos is the elevated mosaicism rate observed at the human pre-implantation stage. Sandalinas and collaborators found that up to 70% of the embryos they studied by FISH were mosaic for some kind of chromosomal abnormality (Sandalinas et al., 2001). Li and co-workers (2005) found that 40% of the embryos diagnosed as aneuploid on day 3 turned out to have a euploid inner cell mass at day 6. Staessen and collaborators found that 17.5% of the embryos diagnosed as abnormal during pre-genetic screening (PGS), and subjected to post-PGD reanalysis, were found to also contain normal cells, and 8.4% were found grossly normal (Staessen et al., 2004). As a consequence, it has been questioned whether the one or two cells studied from an embryo are actually representative of the complete embryo, and whether viable embryos are not being discarded due to the limitations of the technique.

The inventors have developed new procedures for conducting pre-implantation genetic diagnosis disclosed herein that address these and other problems.

### SUMMARY OF THE INVENTION

Embodiments disclosed herein include:

A method of pre-implantation genetic diagnosis comprising the steps of:
(a) contacting one or more isolated blastomeres excised from an in vitro embryo with one or more first probes directed against at least one chromosome of the blastomere;
(b) causing the one or more first probes to bind to the at least one chromosome;
(c) contacting the one or more isolated blastomeres with a second probe directed against a hCG or subunit thereof;
(d) assessing the binding of the one or more first probes in the one or more blastomeres to provide an index of normality of the embryo; and
(e) assessing the binding of the second probe in the one or more blastomeres to provide an index of implantation potential of the embryo.

The invention includes in additional embodiments various kits for performing the methods of the invention.

### DETAILED DESCRIPTION OF THE INVENTION

The present inventors have identified that use of a biological marker or probe (such as an antibody or antibody fragment, m-RNA-FISH, a DNA molecule, etc.) against total-hCG, or a particular form thereof, such as hyperglycosylated-hCG, and combining information pertaining to binding of the biological marker to blastomeric material, with information pertaining to the binding of chromosomal probes for chromosomal abnormalities, that one can evaluate whether blastomeres have a high degree of implantation capability with low genetic abnormality capacity. The biological marker and probes may both be labeled with detection-enhancing material such as a fluorescent label.

A probe or biological marker generally includes a chemical, biological, histochemical, cytochemical, immunological, or other substance or reagent that binds a target moiety in a molecule, a subcellular organelle, a subcellular structure, a cell, and the like. A probe or biological marker has a high affinity for the specific target against which it is directed, and low or nonexistent affinity for other moieties in a cell or subcellular component.

An index of normality of an embryo, and related terms and phrases, relate in general to a determination, or an evaluation, of the state of ploidy of the embryo. In general, with respect to a particular autosomal chromosome pair, or with respect to a unitary X chromosome and a unitary Y chromosome of a normal male cell, departure from diploidy of an autosomal chromosome or of an X chromosome in a female, or from haploidy of an X chromosome and a Y chromosome in a male, is considered an aneuploid state, and accordingly not normal.

An index of implantation potential, and related terms and phrases, relate in general to a determination, or an evaluation, based on the outcome of a procedure disclosed herein, of the likelihood that an embryo will successfully implant within the lining of the uterus when the embryo is transferred thereto.

Thus there is disclosed a method for determining the implantation potential of an embryo, said method comprising the steps of: (a) depositing one or more isolated blastomere on a slide; (b) treating one or more isolated blastomeres with an antibody, antibody fragment, or biological marker directed against hCG and/or hyperglycosylated hCG; (c) treating the one or more isolated blastomeres with one or more chromosome probes designed to detect chromosomal abnormalities; and (d) determining whether the blastomere is indicative of an embryo with high implantation potential and low abnormality potential from the binding of said antibody and chromosome probes.

Methods of preparing and using antibodies including preparation of labeled antibodies are disclosed, by way of nonlimiting example, in “Antibodies: A Laboratory Manual” (Harlow E and Lane D, Cold Spring Harbor Laboratory Press, Cold Spring Harbor, N.Y. (1988)). Anti-hCG antibodies are available, for example, from HyTest Ltd., Finland; Abcam, Inc., Cambridge, Mass.; Sigma-Aldrich, St. Louis, Mo.; and from other suppliers. Antibody B152 directed against hyperglycosylated forms of hCG is described at least in Birken (Tumor Biology 2005; 26:131-141). Antibodies may be labeled with fluorescent labels to create marker molecules suitable for use in FISH assays using fluorescent labeling reagents, and methods for their use, that are widely known to workers of skill in the field of the invention. Such methods and labels are available at least from Pierce Chemical Co., Rockford, Ill.; and Molecular Probes, a division of Invitrogen, Carlsbad, Calif. Nucleic acid probes suitably labeled for use in FISH are likewise prepared by use of labeled mononucleoside triphosphates or their derivatives in enzyme catalyzed nucleic acid synthetic procedures, or by chemical synthesis. These procedures are widely known to workers of skill in the field of the invention. In particular, nucleic acid probes directed at detectable portions of various chromosomes, useful in establishing normality or abnormality of an embryo are widely known in the field of the invention. Examples include probes that are used in Aneuvysion™ or Urovysion™ (Vysis, Inc., a subsidiary of Abbott Laboratories, Downers Grove, Ill.). Paired XY FISH probes include a dual-color FISH using the chromosome enumeration probe cocktail CEPX spectrum green/CEPY [alpha-satellite] spectrum orange (Vysis/Abbott Diagnostics; Baar, Switzerland). A dual Y-Y probe is also available with two different Y-chromosome probes CEPY satellite (spectrum orange) and CEPY III-satellite (spectrum aqua) (Vysis/Abbott).

FISH kits comprising multicolor DNA probes are commercially available. For example, AneuVysion® Multicolor DNA Probe Kit sold by the Vysis division of Abbott Laboratories, is designed for in vitro diagnostic testing for abnonnalities of chromosomes 13, 18, 21, X and Y in amniotic fluid samples via fluorescence in situ hybridization (FISH) in metaphase cells and interphase nuclei. The AneuVysion® Assay (CEP 18, X, Y-alpha satellite, LSI 13 and 21) Multi-color Probe Panel uses CEP 18/X/Y probe to detect alpha satellite sequences in the centromere regions of chromosomes 18, X and Y and LSI 13/21 probe to detect the 13q14 region and the 21q22.13 to 21q22.2 region. The AneuVysion kit is useful for identifying and enumerating chromosomes 13, 18, 21, X and Y via fluorescence in situ hybridization in metaphase cells and interphase nuclei obtained from amniotic fluid in subjects with presumed high risk pregnancies. The combination of colors emitted by the tags is used to determine whether there is a normal chromosome numbers or trisomy.

The UroVysion® kit by the Vysis division of Abbott Laboratories is designed to detect chromosomal abnormalities associated with the development and progression of bladder cancer by detecting aneuploidy for chromosomes 3, 7, 17, and loss of the 9p21 locus via fluorescence in situ hybridization in urine specimens from persons with hematuria suspected of having bladder cancer. The UroVysion Kit consists of a four-color, four-probe mixture of DNA probe sequences homologous to specific regions on chromosomes 3, 7, 9, and 17. The UroVysion probe mixture consists of Chromosome Enumeration Probe (CEP) CEP 3 SpectrumRed, CEP 7 SpectrumGreen, CEP 17 SpectrumAqua and Locus Specific Identifier (LSI 9p21) SpectrumGold.

Chromosome enumeration probes based on centromeric probes for several chromosomes are available from Genzyme Corp., Cambridge, Mass.

Kits for labeling DNA probes for use in FISH are available from Mirus Bio Corp., Madison, Wis. Labels include Cy™3, fluorescein, rhodamine and biotin.

FISH procedures and protocols are described, by way of nonlimiting example, in “Introduction to Fluorescence In Situ Hybridization: Principles and Clinical Applications” 1st edition, Andreef M and Pinkel D (eds.), Wiley-Liss, New York, N.Y. (1999.

An example of a procedure for conducting a FISH analysis on fetal cells is given in Mergenthaler et al. (J. Histochem. Cytochem., 53 (3): 319-322, 2005).

In order to establish indicia for implantation potential, a large number of in vitro fertilized embryos destined for attempted uterine implantation are subjected to analysis. From each embryo, at least one blastomere is isolated in such a way as to preserve the viability of the embryo. The isolated blastomeres are contacted with a labeled antibody directed against hCG or a subunit thereof, as well as with a labeled antibody directed against hyperglycosylated hCG. The respective antibodies are labeled with different fluorophores, such as with Cy™3 and Cy™5, to permit simultaneous evaluation of the extent of labeling. After appropriate rinsing away of excess antibody, the blastomere is examined by microscopy in an immunofluorescence protocol, and the extent of fluorescent labeling by anti-total-hCG as well as by anti-hyperglycosylated hCG is established. Optimally, in order to provide an internal standard for fluorescence yield, a constitutively invariant protein such as beta actin, or a glycolytic enzyme, is simultaneously analyzed by fluorescent antibody labeling, using a third distinguishable fluorescent label. The embryo is transferred in utero and the outcome concerning implantation and fetal development is followed. These data are correlated in a multivariate statistical analysis with the implantation results obtained for each respective embryo from which the at least one blastomere was obtained. The correlations provide a set of standards or criteria against which subsequent analysis performed on unknown embryos may be compared to establish a potential for successful implantation.

In order to establish indicia for the potential for normality or abnormality, a large number of in vitro fertilized embryos destined for attempted uterine implantation are subjected to analysis. A nonlimiting example of analysis for normality is an evaluation of the euploidy or aneuploidy of the blastomere. From each embryo, at least one blastomere is isolated in such a way as to preserve the viability of the embryo. The isolated blastomeres are contacted with at least one labeled nucleic acid probe targeting a nucleic acid sequence characteristic of a particular chromosome. In various embodiments a plurality of probes is employed, including probes having different labels on them to permit multiplexing the FISH analysis. Multiplexing may be directed to the same chromosome, thus affording redundancy in the analysis for that particular chromosome, or to a different chromosome, thus providing results concerning the state of ploidy for more than one chromosome at the same time. If necessary or desired to have standardizations for comparison, suitable parental cells obtained from the mother and/or the father of the embryo may be subjected to parallel chromosomal probing. After appropriate rinsing away of excess probe, the blastomere is examined by microscopy in a FISH protocol, and the extent of fluorescent labeling by the labeled probe is established. These data are correlated in a multivariate statistical analysis with the normality results obtained from each respective embryo from which the at least one blastomere was obtained. The correlations provide a set of standards or criteria against which subsequent analysis performed on unknown embryos may be compared to establish a potential for normality or abnormality.

In an exemplary illustration of evaluating an embryo for its potential for successful implantation and development based on the state of ploidy of chromosome 18, the following steps may be performed:

1. Isolate one or more blastomeres from a blastocyst stage of an embryo, maintaining the blastomeres throughout in suitable conditions;

2. Contact the one or more blastomeres with a labeled chromosome probe for chromosome 18;

3. Optionally include cells from one or both parents of the embryo and contact the parental cells with the same labeled chromosome probe for chromosome 18;

4. Perform a nucleic acid hybridization protocol on the one or more blastomeres and on the parental cells if present;

5. Optionally contact the one or more blastomeres with a labeled antibody that determines total hCG in the cell;

6. Contact the one or more blastomeres with a labeled antibody that determines hyperglycosylated hCG in the cell;

7. Optionally contact the one or more blastomeres with a labeled antibody directed against cellular beta actin or a suitable glycolytic enzyme;

8. Analyze the resulting cellular preparation by fluorescence immunohistochemistry and microscopic FISH analysis;

9. Determine a potential for successful embryonic implantation from the results of the hyperglycosylated hCG analysis;

10. Determine a result for euploidy or aneuploidy for chromosome 18; and

11. Evaluate whether the embryo from which the one or more blastomeres were isolated is suitable for implantation.

### STATEMENT REGARDING PREFERRED EMBODIMENTS

While the invention has been described with respect to preferred embodiments, those skilled in the art will readily appreciate that various changes and/or modifications can be made to the invention without departing from the spirit or scope of the invention as defined by the appended claims.

What is claimed is:

1. A method of pre-implantation genetic diagnosis comprising the steps of:
(a) contacting one or more isolated blastomeres excised from an in vitro embryo with one or more first probes directed against at least one chromosome of the blastomere;
(b) causing the one or more first probes to bind to the at least one chromosome;
(c) contacting the one or more isolated blastomeres with a second probe directed against a hCG or subunit thereof;
(d) assessing the binding of the one or more first probes in the one or more blastomeres to provide an index of normality of the embryo; and
(e) assessing the binding of the second probe in the one or more blastomeres to provide an index of implantation potential of the embryo.

2. The method described in claim 1 wherein the second probe is an antibody or antibody fragment.

3. The method described in claim 2 wherein the antibody or antibody fragment is specifically directed against hyperglycoslated hCG.

4. The method described in claim 1 wherein the one or more first probes targets one or more chromosomes known to be aneuploid in a disease or syndrome.

5. The method described in claim 4 wherein a first probe comprises a nucleic acid.

6. The method described in claim 1 wherein at least one of a first probe and a second probe comprises a label.

7. The method described in claim 6 wherein the label is a fluorescent label.

8. The method described in claim 7 wherein the assessing includes fluorescence analysis of the one or more probed blastomeres.

9. A kit comprising one or more first probes directed against at least one chromosome and a second probe directed against a hCG or subunit thereof disposed in at least one container.

10. The kit described in claim 9 wherein the second probe is an antibody or antibody fragment.

11. The kit described in claim 10 wherein the antibody or antibody fragment is specifically directed against hyperglycoslated hCG.

12. The kit described in claim 9 wherein the one or more first probes targets one or more chromosomes known to be aneuploid in a disease or syndrome.

13. The kit described in claim 12 wherein the one or more first probes comprises a nucleic acid.

14. The kit described in claim 9 wherein at least one of a first probe and a second probe comprises a label.

15. The kit described in claim 14 wherein the label is a fluorescent label.
